(19) United States
(12) Patent Application Publication
(10) Pub. No.: US 2016/0038926 A1
(43) Pub. Date: Feb. 11, 2016

(54) METAL NANO-CATALYSTS IN GLYCEROL AND APPLICATIONS IN ORGANIC SYNTHESIS

(71) Applicants: CENTRE NATIONAL DE LA RECHERCHE SCIENTIFIQUE (CNRS), PARIS Cedex 16 (FR); UNIVERSITÉ PAUL SABATIER TOULOUSE 3, TOULOUSE Cedex 9 (FR)

(72) Inventors: Montserrat GOMEZ, Toulouse (FR); Emmanuelle TEUMA, Auzeville Tolosane (FR); Isabelle FAVIER, Toulouse (FR); Faouzi CHAHDOURA, Toulouse (FR)

(21) Appl. No.: 14/654,061

(22) PCT Filed: Dec. 20, 2013
(86) PCT No.: PCT/FR2013/053215

§ 371 Date: Jun. 19, 2015

(30) Foreign Application Priority Data
Dec. 21, 2012 (FR) 1262533

(51) Int. Cl. B01J 35/00; B01J 31/06; B01J 31/28; B01J 23/44; B01J 23/46; B01J 23/72; C07C 41/20; C07C 45/62; C07C 67/303; C07D 209/08; C07D 211/62; C07C 209/60; C07D 295/135; C07D 295/096; C07C 319/18; C07D 307/79; C07D 307/89; C07D 401/06; C07C 45/64; C07D 249/04; C07D 403/14; C07D 307/84; C07D 493/04; C07D 405/06; C07D 493/14; C07D 493/18; C07D 211/94; C07D 401/04; C07C 5/03; B01J 31/02

(52) U.S. Cl. CPC B01J 35/0013; B01J 31/0202; B01J 31/06; B01J 31/0267; B01J 31/0271; B01J 31/28; B01J 23/44; B01J 23/464; B01J 23/72; C07C 41/20; C07C 45/62; C07C 67/303; C07D 209/08; C07D 211/62; C07C 209/60; C07D 295/135; C07D 295/096; C07C 319/18; C07D 307/79; C07D 307/89; C07D 401/06; C07C 45/64; C07D 249/04; C07D 403/14; C07D 307/84; C07D 493/04; C07D 405/06; C07D 493/14; C07D 493/18; C07D 211/94; C07D 401/04; C07C 5/03; C07C 2101/14; C07C 2531/28; C07C 2531/02

(57) ABSTRACT

A catalytic system which is a suspension in glycerol of metal nanoparticles in at least one transition metal. The suspension also includes at least one compound stabilizing the metal nanoparticles, soluble in glycerol. The suspensions are obtained directly in glycerol. These are stable systems that can catalyse a reaction from an organic substrate, with high yields and activity, and excellent selectivity. Additionally, the use of the catalytic system for performing organic transformations such as hydrogenation or coupling reactions (formation of C—C, C—N, C—O, C—S . . . bonds), and for synthesizing polyfunctionnal molecules, in a single reactor, by multi-step, sequential or cascade reactions.

The present invention relates to the field of catalytic systems comprising metal nanoparticles which are intended to be employed in organic synthesis.

A subject matter of the present invention is a composition comprising metal nanoparticles in suspension in glycerol, and also a process for obtaining such a suspension. Another subject matter of the invention is the use of said suspension of metal nanoparticles as catalytic system in organic synthesis reactions.

The design of environmentally friendly processes is one of the major objectives of current research and in particular, since the start of the 21^{st} century, in the context of the European directives drawn up during the Göteborg summit in 2001. The fine chemicals industry (pharmaceutical industry, agrochemical industry), which uses large volumes of conventional organic solvents of petrochemical origin, is particularly affected. It is henceforth desired to reduce and to eliminate the use of environmentally harmful substances and the generation of byproducts, by novel chemical processes and sustainable synthesis routes. It is a matter of preventing the production of waste rather than investing in its removal, which catalysis makes possible.

In order to do this, chemists are prompted by several concerns relating to synthesis processes and in particular the choice of the catalysts and solvents. It is necessary to favor the use of catalysts, in order to render the reactions as selective as possible. When this is possible, it is necessary to abandon the use of additives and to work under mild conditions (low temperatures, low pressures, and the like). During the last ten years, in agreement with the 12 principles of Green Chemistry, novel solvents have been used: water, ionic liquids, supercritical CO₂ and fluorinated solvents. In particular, processes using nontoxic and biodegradable solvents, exhibiting a low volatility, have appeared as appropriate alternatives to volatile organic solvents (VOCs).

Homogeneous catalysis makes it possible to work under mild conditions, which makes it a suitable means for synthesis in fine chemistry which requires moderate temperatures and low pressures. In the context set out above, and because of the large volumes of solvents used for the syntheses, a system which observes the criteria of green chemistry is desired. While it is necessary to replace conventional organic solvents with nonpolluting solvents, it is also desired to immobilize the catalytic phase. This makes it possible, on the one hand, to reduce the consumption of metals and ligands, which are expensive, and, on the other hand, to reduce the content of metal in the products obtained, in order to improve the environmental impact. The product must be as pure as possible, with a low metal content, on the ppm scale, indeed even ppb scale.

Catalytic systems based on metals in ionic liquids have already been developed by the inventors. The catalysts are either molecular (nickel, ruthenium, rhodium, platinum, iridium, palladium or molybdenum complexes) or colloidal (palladium, rhodium or ruthenium nanoparticles). Catalytic systems based on metal nanoparticles in ionic liquids are described, for example, in the documents WO2009/024312 and WO2008/145836, and their use in organic catalysis in WO2008/145835. The use of these solvents experiences limitations on the industrial scale: high price, lack of data relating to their toxicity and low biodegradability.

With regard to water, its use as solvent is restrictive insofar as the reactants and also the products of the reactions are organic compounds with little or no solubility in water. Its use is also limited due to the instability of the catalysts.

Recently, interest has arisen in the use of solvents resulting from biomass as replacement for those resulting from petroleum and more particularly of glycerol, which might represent an economically advantageous alternative for industrial applications. This is because this inexpensive compound is the byproduct obtained in the production of biodiesel and in conversions of cellulose or lignocellulose. Since the first work published in 2006 using glycerol as solvent, numerous papers have been published targeted at applications in biocatalysis, but some only relate to organometallic catalysis, involving molecular complexes. Mention may be made, for example, of:
the synthesis of diarylalkenes through the diarylation of acrylates, catalyzed by palladium with iodoarenes, using an aminopolysaccharide as ligand (for a selected contribution, see: S. B. Park and H. Halper, Org. Lett., 2003, 5, 3209);
the telomerization of butadiene with carbon dioxide, catalyzed by palladium, to form δ-lactones (A. Karam, N. Villandier, M. Delample, C. K. Koerkamp, J.-P. Douliez, R. Granet, P. Krausz, J. Barrault and F. Jérôme, Chem. Eur. J., 2008, 14, 10196);
the hydrogenation of styrene in pure glycerol by using [RhCl(TPPTS)₃] and Pd/C as catalysts (K. Tarama and T. Funabiki, Bull. Chem. Soc. Jpn., 1968, 41, 1744, and also A. Wolfson, C. Dlugy and Y. Shothland, Environ. Chem. Lett., 2007, 5, 67);
enantioselective hydrogenation with catalysts based on Ru/(S)-BINAP;
the enantioselective reduction of the C═C double bond of conjugated esters, with NaBH₄ as reducing agent (L. Aldea, J. M. Fraile, H. Garcia-Marin, J. I. Garcia, C. I. Herreria, J. A. Mayoral and I. Pérez, Green Chem., 2010, 12, 435);
the transfer of hydrogen from several ketones and aldehydes using catalysts based on iridium and on ruthenium (E. Farnetti, J. Kaspar and C. Crotti, Green Chem., 2009, 11, 704, and A. Wolfson, C. Dlugy, Y. Shothland and D. Tavor, Tetrahedron Lett., 2009, 50, 5951);
the cycloisomerization starting from (Z)-enynols by palladium complexes comprising hydrophilic ligands (J. Francos and V. Cardieno, Green Chem., 2010, 51, 6772);
the synthesis of 1,4-dihydropyridines in glycerol by a catalyst based on cerium (A. V. Narsaiah and B. Nagaiah, Asian J. Chem., 2010, 22, 8099).

The study of the state of the art in this field shows that, despite a few rare encouraging preliminary results, little research has been carried out to date in order to be able to make use of the potentialities of glycerol as solvent for catalytic reactions using organometallic compounds and none using preformed metal nanoparticles as catalytic precursors.

First, research has been carried out into a method of obtaining a catalytic system, based on glycerol comprising nanoparticles comprising a metal, this system having to be stable, that is to say without observation of agglomeration phenomena, which are frequent when nanoparticles are handled, particularly in solution, which might result subsequently in the deactivation of the catalyst. The use of such a system has the aim in particular of making possible the recycling and the easy reuse of the catalytic phase.

Secondly, the compatibility of the catalyst and of the solvent has to be confirmed as it may be expected that the glycerol will have an undesirable reactivity due to the alcohol functional groups which it carries. In addition, as a result of its viscosity, it has appeared essential to operate at temperatures greater than ambient temperature in order to avoid limitations by mass transfer. A study of the stabilizers compatible with glycerol thus assumes critical importance, in order to apply the solvent in the selective processes concerned.

Unexpectedly, we have found that glycerol (propane-1,2,3-triol), which can result from biomass, represents an appropriate solvent for the stabilization of nanoparticles of transition metals, in the presence of stabilizing ligands or polymers. It has been found that it is possible to synthesize metal nanoparticles directly in glycerol and that these suspensions are stable and exhibit a high activity and a high selectivity for catalytic processes. The colloidal solutions (suspensions) obtained are indeed composed of metal nanoparticles which are small in size (less than 20 nm) and well dispersed in the glycerol. This control of the structural characteristics is acquired by virtue of the preparation of the nanoparticles by the chemical route and of the choice of a stabilizing compound for nanoparticles in glycerol which is suited to the reaction medium. In addition, the suspensions obtained can be stored with retention of their characteristics, so that it is possible to market them. Finally, it is found that it is easy to recycle the catalytic phase.

More specifically, the present invention relates to a catalytic composition, which consists of a suspension in glycerol of metal nanoparticles comprising at least one transition metal, said suspension also comprising at least one glycerol-soluble stabilizing compound which stabilizes said metal nanoparticles.

The expression “catalytic solution” is generally employed in the field concerned to denote a composition as defined above. However, the expression “catalytic system” will be preferred to it subsequently. Such a system comprises a compound acting as catalyst for a specific reaction and a solvent suited to the implementation of said reaction. Metal nanoparticles is understood to mean particles the size of which can vary from 1 to 100 nanometers. The size of the nanoparticles is determined by standard structural characterization techniques. Transmission electronic microscopy (TEM) makes it possible, for example, to characterize the metal nanoparticles and to obtain direct visual information on the size, morphology, dispersion, structure and arrangement of the nanoparticles. The nanoparticles are described as metal nanoparticles insofar as they are formed of atoms of at least one metal, which is optionally oxidized, as will be described in detail later.

According to an advantageous characteristic of the invention, said metal nanoparticles have a mean size of less than 20 nm, which gives them effective catalytic behaviors. Preferably, their size is less than 10 nm and more preferably it is between 1 nm and 5 nm. The mean size of the particles according to the invention is determined from the measurement of a batch of 2000 or more particles, using a counting software based on shape recognition.

The methodology for the synthesis of colloidal suspensions in glycerol of metal nanoparticles can be applied to various transition metals in the zero or positive oxidation state, so that the system according to the invention can be obtained for nanoparticles of various metals. According to an advantageous embodiment of the invention, said nanoparticles comprise a metal having a zero oxidation state chosen from the transition metals from Groups VI to XI. According to another advantageous embodiment of the invention, said nanoparticles comprise an oxide of a transition metal having a given oxidation state, or a mixture of oxides of a transition metal having different oxidation states, said metal being chosen from the metals of the first transition series, such as, in particular, manganese, iron, cobalt, nickel or copper.

According to a preferred embodiment of the invention, said nanoparticles comprise a metal chosen from palladium, rhodium, ruthenium and copper. In particular, the invention relates to nanoparticles of palladium (PdNP), rhodium (RhNP) and copper(I) oxide (Cu₂ONP), which are synthesized in glycerol.

The system which is a subject matter of the present invention comprises a stabilizing compound which can be a polymer or a ligand and which is soluble in glycerol. It is known that the nanoparticles of transition metals are naturally not very stable and have a strong tendency to agglomerate, thus losing their nanometric nature. This aggregation normally results in the loss of the properties related to their colloidal state and is generally reflected in catalysis by a loss of activity and problems of reproducibility. The stabilization of the metal nanoparticles and thus the maintenance of their size, shape and dispersion is a decisive condition for their catalytic properties. Various stabilizing compounds, which are described, for example, in US 2006/115495, are known. However, the nature of the solvent employed in the present system has resulted in the nature of this stabilizer being adjusted.

It is possible to choose a stabilizing ligand from glycerol-soluble phosphines. In this case, preference will be given to the sodium salt of tris(3-sulfophenyl)phosphine (abbreviated to TPPTS). This compound, which is soluble in water, has proved to be also soluble in glycerol and to be capable of fully playing its stabilizing role. In this case, the molar ratio of the ligand to the metal in the nanoparticles can advantageously be between 0.1 and 2.0 and preferably between 0.2 and 1.0.

It is also possible to choose the stabilizing compound from glycerol-soluble polymers. In this case, preference will be given to poly(N-vinylpyrrolidone) (PVP). This compound has proved to be soluble in glycerol and, in doing this it fully plays its stabilizing role. Advantageously, the molar ratio of the monomer of said polymer to said metal is between 1 and 100 and preferably between 15 and 40.

According to a particularly advantageous characteristic of the catalytic system which is a subject matter of the present invention, said transition metal is at a concentration in the glycerol of between 10⁻¹ mol/l and 10⁻⁴ mol/l, preferably close to 10⁻² mol/l.

Another subject matter of the present invention is a process for obtaining a catalytic system consisting of a suspension in glycerol of metal nanoparticles as is described above, the process comprising the stages consisting essentially in:

a) introducing, into a reactor, i) an amount of glycerol, ii) at least one precursor compound of a transition metal, and iii) at least one glycerol-soluble stabilizing compound which stabilizes said metal nanoparticles;

b) placing this reaction mixture under a pressure of a reducing gas of between 10⁵ Pa and 5×10⁵ Pa (1 bar and 5 bar), at a temperature of between 30° C. and 100° C., and allowing reaction to take place until the precursor has completely decomposed and a suspension of nanoparticles of said metal compound has formed.

According to one embodiment of the process according to the invention, said precursor can be a salt of said transition metal, such as a halide, an acetate, a carboxylate or an acetylacetonate, or an organometallic complex of a transition metal or also an oxide of said metal. According to a preferred embodiment, said precursor is an organometallic complex of said transition metal. Said transition metal can be chosen from the elements of Groups VI to XI. Preferably, said transition metal is copper, palladium, rhodium or ruthenium.

It has been seen that the stabilizing compound can be a polymer or a ligand. According to a specific embodiment, said stabilizing compound is chosen from glycerol-soluble phosphines. Preferably, the sodium salt of tris(3-sulfophenyl)phosphine (TPPTS) is chosen. In this case, the molar ratio of said ligand to said metal (that is to say, with the metal precursor) is advantageously between 0.1 and 2.0. It is preferably between 0.2 and 1.0. For example, palladium and rhodium metal nanoparticles (MNP) can be prepared by decomposition of salts or organometallic complexes (Pd(OAc)₂ or [RhCl(CO)₂]₂) in the presence of TPPTS present in a proportion of 0.3 to 1 equivalent with respect to the metal.

According to another specific embodiment of the invention, the stabilizing compound is chosen from glycerol-soluble polymers, preferably poly(N-vinylpyrrolidone) (PVP). In this case, the molar ratio of the monomer of said polymer to said metal (that is to say, the metal precursor) can advantageously be between 1 and 100. It is preferably between 15 and 40. For example, copper(I) oxide nanoparticles, Cu₂ONP, can be prepared by decomposition of copper(II) acetate in the presence of PVP (average molecular mass 10 000 g/mol) with a monomer/Cu ratio of 20.

According to an advantageous characteristic of the process which is the subject matter of the invention, the metal precursor is introduced into the reactor at a concentration between 10⁻¹ mol/l and 10⁻⁴ mol/l. Preferably, it is employed at a concentration close to 10⁻² mol/l.

The other characteristics of the process according to the invention are preferably as follows:
the pressure of the reducing gas is obtained with molecular hydrogen at 3 bar (3×10⁵ Pa),
the temperature is between 30° C. and 100° C. and is preferably approximately 60° C.,
the duration of the reaction is between 5 hours and 20 hours.

The colloidal systems thus obtained were characterized by transmission electron microscopy (TEM) owing to the negligible vapor pressure of glycerol under analytical conditions. It should be emphasized that these analyses can be carried out directly on the suspension, without it being necessary to isolate the solid phase, owing to the negligible vapor pressure of the solvent, glycerol. This methodology for the analysis of samples is particularly advantageous for liquid-phase catalytic reactions (referred to as “homogeneous catalysis”). The TEM images show that the nanoparticles are well dispersed in the glycerol in the presence of stabilizing compounds and that their size is small and homogeneous. This will make possible high catalytic activities and selectivities during chemical transformations in glycerol.

A stable catalytic system is thus available, which system can be used directly to catalyze a reaction starting from an organic substrate, the solvent of which is glycerol. As a result of its physicochemical properties, it henceforth represents a solvent of choice for liquid-phase reactions. This is because glycerol has a high boiling point with a broad range of temperatures in the liquid state (17.8° C.-290° C.), its vapor pressure is insignificant (namely less than 1 mmHg at 20° C.), its dielectric constant is high (which will make possible better solubility in particular of polar compounds) and its toxicity is virtually zero: LD₅₀ (oral in rats)=12 600 mg/kg. Its environmental impact is negligible in comparison with that of the usual volatile organic solvents used in fine chemistry.

The system described above has proved to be a catalytically active system, with a high activity and high yields and an excellent selectivity. This is why the present invention also has as subject matter a synthesis process starting from an organic substrate employing, as catalytic system, said suspension of metal nanoparticles in glycerol.

There is thus claimed the use of a catalytic system (comprising a solvent and a catalyst) for catalyzing an organic synthesis reaction starting from a substrate, in which: j) said substrate is brought into contact with said catalytic system comprising at least one metal capable of catalyzing said reaction, at a temperature of between 30° C. and 100° C., then jj), at the end of the reaction, the products obtained and the catalytic system are separated. In this procedure, the metal catalyst is in the form of preformed nanoparticles in suspension in the glycerol. The reaction takes place under mild conditions, with moderate temperatures and a pressure which can vary as a function of the catalytic process (less than 5×10⁵ Pa).

The applications relate to organic transformations which are of interest in the field of fine chemistry, in particular for the pharmaceutical sector, such as coupling reactions (formation of C—C, C—N, C—O, C—S, and the like, bonds) or hydrogenation reactions, and also their applications in multistage processes (cascade or sequential reactions).

At the end of the reaction, the products formed are extracted with an organic solvent, for example dichloromethane, which is easy as glycerol exhibits low miscibility with organic solvents (this being an additional argument in favor of its use). The catalytic phase, namely the suspension of metal nanoparticles in glycerol, then remains. It is then easy to recycle it, by evaporating, under vacuum, the traces of the extraction solvent. It is possible to again use it for a new reaction and this up to 10 and more times, whereas this is inapplicable to catalysts in an organic medium. The use of glycerol as solvent for catalytic reactions corresponds to the definition of an environmentally friendly solvent according to the principles of Green Chemistry, by making possible easy extraction of organic products and effective immobilization of the catalyst in the glycerol phase, which greatly facilitates the recycling thereof.

Thus, particularly advantageously according to the invention, once the products are extracted, said catalytic system is recycled by subjecting it to a reduced pressure (approximately 10³ Pa), for example for 30 minutes, and stages j) and jj) are repeated at least once, preferably 5 times and more preferably more than 10 times, with identical or different substrates and reactants.

In accordance with a specific use of a catalytic system according to the invention, a hydrogenation reaction catalyzed by a catalytic system comprising rhodium nanoparticles in suspension in glycerol is carried out. For example, metal nanoparticles obtained as indicated above are effective catalytic systems for the selective hydrogenation of the C═C double bond of monosubstituted alkenes, such as styrene and derivatives, for 1,2-disubstituted and 1,1-disubstituted olefins, or also for trisubstituted cyclic alkenes. These reactions are carried out under mild conditions (10⁵-3×10⁵ Pa H₂ with catalyst contents of 0.1 mol %). The yields are in all cases between 85% and 99%. The system can be recycled without loss of activity (at least 5 times).

In accordance with another specific use of a catalytic system according to the invention, a reaction is carried out in which the formation of a C—N or C—S bond is catalyzed by a catalytic system comprising copper(I) oxide nanoparticles in suspension in glycerol. Mention may be made, for example, of the direct coupling of primary or secondary amines with iodobenzene derivatives, in a basic medium, catalyzed by Cu₂ONP, which results in the formation of secondary or tertiary amines respectively, with yields ranging from 92% to 99%. This catalyst is also effective for the coupling of thiophenols, producing the corresponding thioethers, with yields of the order of 90%, under the same operating conditions.

In accordance with another specific use, a reaction is carried out in which the formation of a C—C bond is catalyzed by a catalytic system comprising palladium nanoparticles in suspension in glycerol. This coupling reaction can, for example, be:
a Suzuki C—C cross-coupling reaction, in which a substrate reacts with a boronic acid derivative; or
a Heck C—C cross-coupling reaction, in which a substrate reacts with an alkene derivative; or
a Sonogashira C—C cross-coupling reaction, in which a substrate reacts with an alkyne derivative.

The palladium nanoparticles have been shown to be very active and chemoselective, in particular for these C—C cross-couplings. The Sonogashira coupling was obtained without it being necessary to add a cocatalyst. The catalytic phase in glycerol can be recycled many times without loss of activity or of yield.

According to yet another specific use, a carbonylative coupling reaction is carried out, in which a substrate carrying a carboxylic acid functional group reacts with an amine derivative, the reaction being catalyzed by a catalytic system comprising palladium nanoparticles in suspension in glycerol according to the invention. The high yield of these reactions makes it possible to carry out several reactions in a single reactor (one-pot reaction), in cascade or sequentially, without having to isolate or purify the intermediate products. It is in particular highly advantageous to carry out one-pot multistage syntheses for the formation of various types of heterocycles.

As is seen with the reaction protocols above, given as nonlimiting examples, the use of the catalytic system according to the invention opens a wide range of application in the field of fine chemistry, since it makes possible the preparation of molecules which are sometimes difficult to access, such as active principles of medicaments used in the pharmaceutical industry. In doing this, the use of volatile organic solvents, generally used in a large amount, is avoided, which is one of the current environmental challenges of the fine chemicals industry.

The catalytic system based on metal nanoparticles in glycerol which forms the subject matter of the invention thus exhibits multiple advantages: it is easy to handle and the separation of the products formed and of the catalytic phase is easy as result of the low miscibility with other organic solvents (hence a saving in time and in the amount of the extraction solvents), implying that the products obtained are not contaminated by metal. Furthermore, the glycerol solvent is inexpensive, nontoxic and nonflammable with a high boiling point and a low vapor pressure (hence the suppression of any traces of solvent in the air). Also, in the presence of glycerol, the catalytic systems are highly selective, which makes it possible to minimize the formation of byproducts (saving in atoms). During organic transformations, the glycerol also makes it possible to use small amounts of metal and to have short reaction times, and low pressures can be applied as result of the good solubility of the gases in this medium. Furthermore, by facilitating the recycling of the catalytic phase, it provides the possibility of using a reduced amount of metal, an important saving in the light of the current prices of the metals (Pd, Ru, and the like). All these characteristics and advantages are perfectly in line with the rules of renewable chemistry.

A better understanding of the present invention will be obtained, and details concerning it will become apparent, by virtue of the description which will be made of one of its alternative embodiments, in connection with the appended figures, in which:

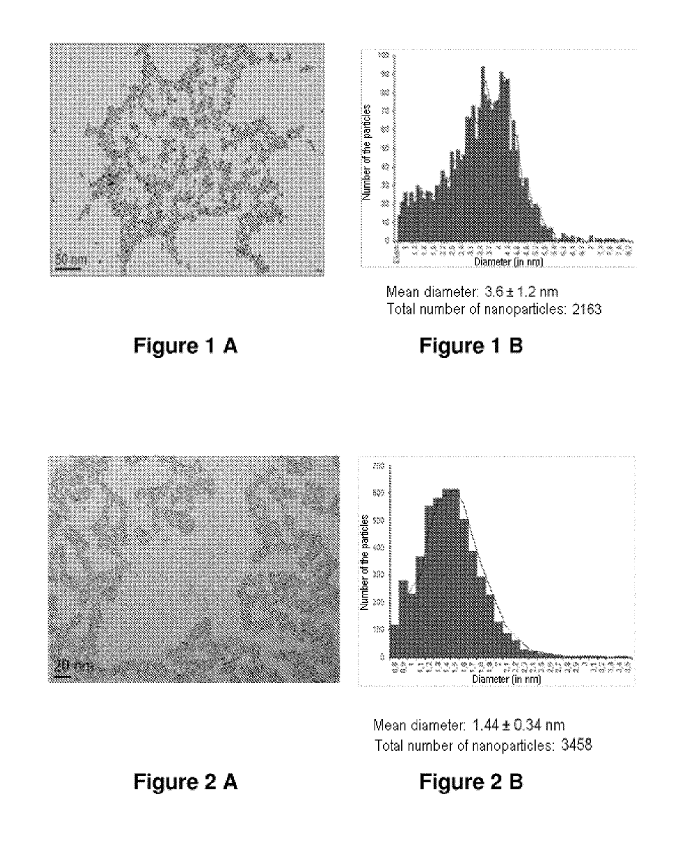

FIG. 1A is a TEM image of palladium nanoparticles prepared according to the invention.

FIG. 1B represents the size distribution of these nanoparticles.

FIG. 2A is a TEM image of rhodium nanoparticles prepared according to the invention.

FIG. 2B represents the size distribution of these nanoparticles.

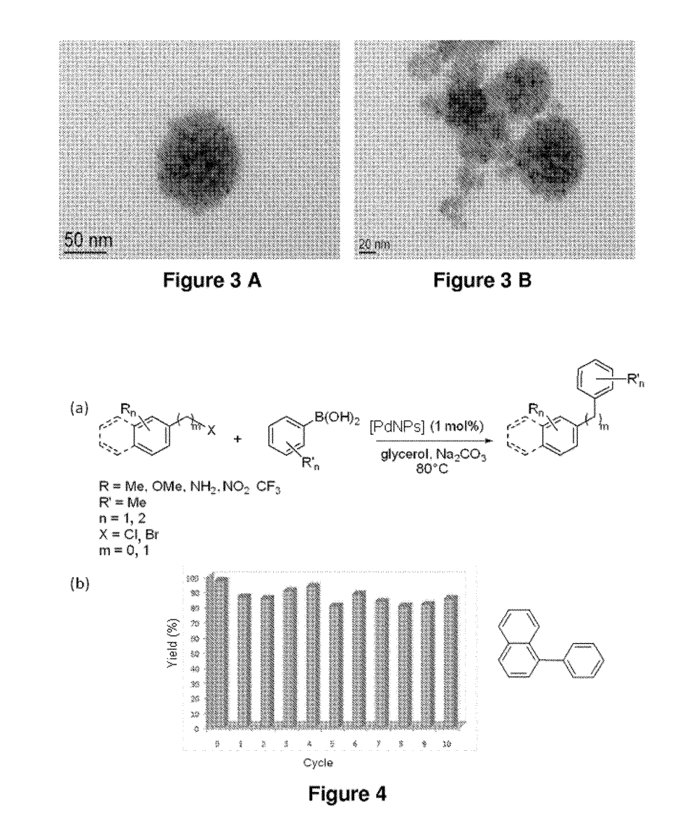

FIGS. 3A and 3B are TEM images of copper(I) oxide nanoparticles prepared according to the invention, at two different scales.

FIG. 4 gives the scheme of the Suzuki cross-coupling reaction (FIG. 4a) and the yields after 10 recyclings of the catalytic phase (FIG. 4b).

### EXAMPLE 1

### Synthesis of Pd and Rh Metal Nanoparticles in Glycerol

The Pd and Rh metal nanoparticles (MNP) were prepared according to the reaction schemes (a1) and (a2) by decomposition of salts or organometallic complexes (Pd(OAc)₂ or [RhCl(CO)₂]₂) in the presence of the TPPTS ligand (1 equivalent with respect to the metal) in pure glycerol, i.e.:
palladium nanoparticles PdNP: 5×10⁻² mmol of Pd(OAc)₂ (11.2 mg) and 1 equivalent of TPPTS (28.4 mg), metal concentration of 10⁻² mol/l;
rhodium nanoparticles RhNP: 5×10⁻² mmol of [RhCl(CO)₂]₂ (9.7 mg) and 1 equivalent of TPPTS (28.4 mg), metal concentration of 10⁻² mol/l.

The precursor, the TPPTS and the glycerol are placed in a Fischer-Porter bottle and heated at 60° C. under a pressure of 3 bar of molecular hydrogen for 18 h.

$$\begin{matrix}{Pd ( OAc )}_{2} + TPPTS {{}^{3 bar {H}_{2}}}_{glycero l , 60 ° C .} PdNP & ( a 1 ) \\ {[ {RhCl ( CO )}_{2} ]}_{2} + TPPTS {{}^{3 bar {H}_{2}}}_{glycerol , 60 ° C .} RhNP & ( a 2 )\end{matrix}$$

After 18 h, complete decomposition of the metal precursor is observed: the initial yellow solutions have become black colloidal solutions. The colloidal systems obtained were characterized by transmission electron microscopy (TEM). Nanoparticles which are well dispersed and homogeneous in size are observed (FIGS. 1A and 2A). The calculated mean diameters are as follows: 3.6 nm for PdNP and 1.4 nm for RhNP (FIGS. 1B and 2B). These analyses were carried out in solution, without isolating the solid phase.

### EXAMPLE 2

### Synthesis of Cu(I) Oxide MNP in Glycerol

The copper(I) oxide nanoparticles Cu₂ONP were prepared by decomposition of copper(II) acetate (5×10⁻² mmol of Cu(OAc)₂) in the presence of PVP (average molecular weight 10 000 g/mol), with a Cu/monomer ratio of 1/20, under the same conditions as those described above (scheme (b)). An orange-colored suspension is obtained after reacting at 100° C. for 18 h.

$${Cu ( OAc )}_{2} + PVP {{}^{3 bar {H}_{2}}}_{glycerol , 100 ° C .} {Cu}_{2} ONP \qquad ( b )$$

The colloidal system obtained was characterized by TEM microscopy. The analysis of the Cu₂ONP nanoparticles shows the formation of nanospheres with a mean diameter of approximately 50 nm (FIGS. 3A and 3B), composed of smaller particles. The analyses were carried out in solution, without isolating the solid phase.

### EXAMPLE 3

### Hydrogenation Reactions Catalyzed by RhNP in Glycerol

Rh nanoparticles obtained as described in example 1 were used to catalyze selective hydrogenation reactions of C═C double bonds of various compounds. The reaction schemes are presented below for the various substrates:

monosubstituted olefins (A and B), 1,2-disubstituted olefins (C), 1,1-disubstituted olefins (D, E) and trisubstituted cyclic alkenes (F). The same experimental protocol was followed for all the substrates. A volume of 0.1 ml of catalytic system (10⁻² mol/l of Rh) formed of preformed rhodium nanoparticles in glycerol is placed in a Fischer-Porter bottle under argon in the presence of 1 mmol of substrate. The reactions are carried out under 1 to 3 bar of molecular hydrogen at between 60 and 100° C. At the end of the reaction, organic products are extracted with dichloromethane (5×3 ml). The organic phase is subsequently filtered through celite, the solvent is evaporated under reduced pressure and the corresponding residue is analyzed by GC-MS and ¹H NMR.

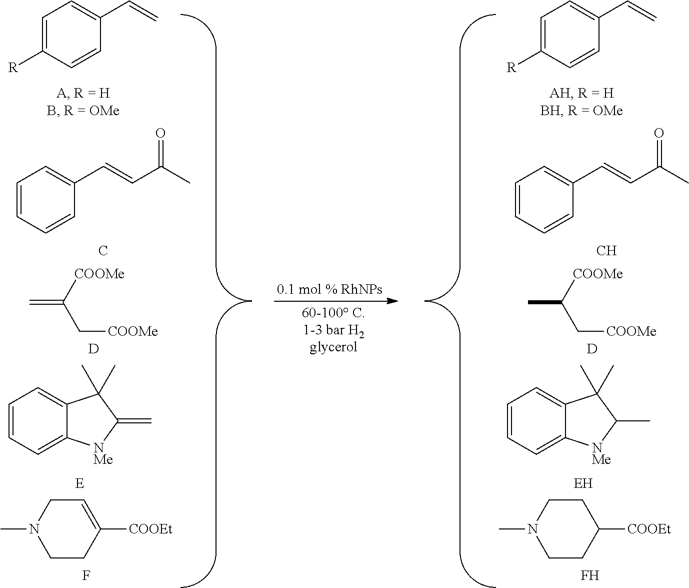

The analyses show that the hydrogenated products are obtained selectively (see scheme above, products AH to FH), with yields of between 85% and 99%.

### Hydrogenation of 4-phenylbut-3-en-2-one and Recycling of the Catalytic Phase:

The hydrogenation of the C═C double bond of 4-phenylbut-3-en-2-one (substrate C) was carried out according to the protocol described above. The reaction was carried out with 0.1 ml of catalytic system formed of rhodium nanoparticles, in the presence of 1 ml of glycerol and of 1 mmol (146 mg) of 4-phenylbut-3-en-2-one. The reaction is carried out at 100° C. under 3 bar of molecular hydrogen. After extraction, the product obtained is analyzed by GC-MS and ¹H NMR. The chromatogram shows the exclusive formation of the CH product (4-phenylbutan-2-one). The weight of final product recovered is 142 mg, i.e. a yield of 95%.

As indicated above, the catalytic system is easily recycled while retaining a high catalytic activity. To do this, at the end of the reaction and once the products have been extracted, the catalytic phase is subjected to a reduced pressure (10³ Pa) for 30 minutes in order to remove all the volatile compounds. A new process can then get underway: the reactants are introduced into the reactor under argon and reacted as described above. The hydrogenation reaction of the substrate C was repeated several times, after recycling the catalytic phase. The CH product obtained was weighed after each cycle. For 7 successive cycles, the weights recovered and the yields are as follows:
first handling 142 mg (95%)
recycling 1: 144 mg (97%) recycling 2: 140 mg (95%)
recycling 3: 141 mg (94%) recycling 4: 139 mg (93%)
recycling 5: 137 mg (92%) recycling 6: 144 mg (97%)

GC/MS of the First Experiment and of the 6 Recyclings

Analytical conditions: 40° C. (2 min)+2 degrees per minute up to 300° C. (5 minutes).

Apparatus: PerkinElmer Clarus 500. Column BPX5 (25 m, diameter 250 μm). Carrier gas helium 15 ml/min. FID and mass detectors. Injector temperature: 250° C. FID temperature: 260° C. Mass detector temperature: 200° C. Retention time: 8.3 minutes.

### EXAMPLE 4

### Formation of C—N and C—S Bonds Catalyzed by Cu₂ONP/Glycerol

The direct coupling of primary or secondary amines with iodobenzene derivatives, catalyzed by Cu₂ONP prepared as illustrated in example 2, was carried out in basic medium according to the schemes below. It results in the formation of secondary or tertiary amines, with yields ranging from 92 to 99%, after reacting at 100° C. for 4 h. The coupling of these iodobenzene derivatives with 4-methylthiophenol made it possible to obtain the corresponding thioether, with a yield of 90%, under the same operating conditions.

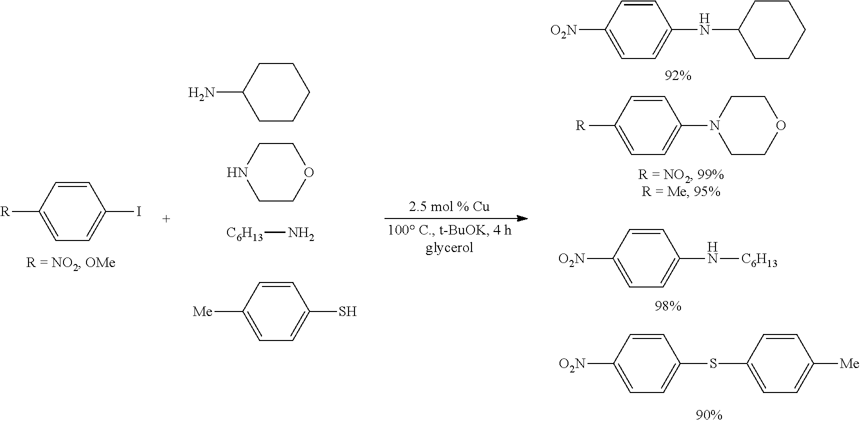

### Experimental Protocol

1 ml of catalytic system (10⁻² mol/l of Cu(I)) formed of preformed Cu₂O nanoparticles in glycerol is placed in a Schlenk tube under argon. A volume of 0.6 mmol of amine derivative or thiol derivative, 1 mmol of t-BuOK and 0.4 mmol of substrate are successively introduced. The reaction is carried out at 100° C. for 4 h. The solution is then cooled to ambient temperature and the products are extracted with dichloromethane (5×3 ml). The organic phase is subsequently filtered through celite, the solvent is evaporated under reduced pressure and the corresponding residue is analyzed by GC-MS and ¹H NMR.

### An Example: Condensation Reaction of Hexylamine and p-Iodonitrobenzene

The following transformation was carried out, according to the preceding experimental protocol.

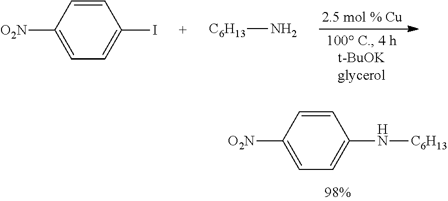

The reaction was carried out with 1 ml of catalytic system formed of Cu₂O nanoparticles in the presence of 0.4 mmol of hexylamine (52.8 μl), 1 mmol of t-BuOK (112 mg) and 0.4 mmol of p-iodonitrobenzene (99 mg). The reaction is carried out at 100° C. for 4 h. The solution is then cooled to ambient temperature and the product is extracted with dichloromethane (5×3 ml). The product is analyzed by GC-MS and ¹H NMR. The chromatogram shows the exclusive formation of the secondary amine product by condensation with formation of a C—N bond. The weight of final product recovered is 87 mg (yield 98%).

GC/MS Analyses

Analytical conditions: 40° C. (2 min)+2 degrees per minute up to 300° C. (5 minutes).

Apparatus: PerkinElmer Clarus 500. Column BPX5 (25 m, diameter 250 μm). Carrier gas helium 15 ml/min. FID and mass detectors. Injector temperature: 250° C. FID temperature: 260° C. Mass detector temperature: 200° C. Retention time: 13.8 minutes.

### EXAMPLE 5

### Cross-Couplings Catalyzed by PdNP in Glycerol

These reactions, which involve the formation of C—C bonds, were carried out with a PdNP catalytic system prepared according to example 1.

### Suzuki C—C Coupling Reaction and Recycling

This scheme is given in FIG. 4(a). The protocol is as follows: 1 ml of catalytic system (10⁻² mol/l of Pd) formed of preformed palladium nanoparticles in glycerol is placed in a Schlenk tube under argon. 1.5 mmol of boronic acid derivative, 2.5 mmol of Na₂CO₃ or t-BuOK and 1 mmol of substrate are then successively introduced. The reaction is carried out at 80-100° C. The solution is then cooled to ambient temperature and the products are extracted with dichloromethane (5×3 ml). The organic phase is subsequently filtered through celite and the solvent is evaporated under reduced pressure. The corresponding residue is analyzed by GC-MS and ¹H NMR. For example, the reaction was carried out with 0.1 ml of catalytic system formed of PdNP nanoparticles in the presence of 0.1 mmol of 1-iodonaphthalene (14.6 μl), 0.15 mmol of phenylboronic acid (18.3 mg) and 0.25 mmol of Na₂CO₃ (26.5 mg) at 100° C. for 12 h. The solution is cooled to ambient temperature and the product is extracted with dichloromethane (5×3 ml). After extraction, the product obtained is analyzed by GC-MS and ¹H NMR. The chromatogram shows the exclusive formation of the cross-coupling product.

The Suzuki cross-coupling reaction described above was carried out in order to obtain 1-phenylnaphthalene. It was repeated 10 times, with the same recycled catalytic phase: once the product has been extracted, the catalytic phase is treated under reduced pressure for 30 minutes. The reactants are then again introduced under argon and reacted as described above. The yields are represented in FIG. 4(b) and are:
first handling 20 mg (98%)
recycling 1: 19 mg (93%) recycling 2: 19.5 mg (95%)
recycling 3: 19.8 mg (97%) recycling 4: 18 mg (88%)
recycling 5: 19 mg (93%) recycling 6: 19.6 mg (96%)
recycling 7: 19.5 mg (95%) recycling 8: 19.8 mg (97%)
recycling 9: 20 mg (98%) recycling 10: 18 mg (88%)
recycling 11: 18.7 mg (91%)

GC/MS of the First Experiment and of the 11 Recyclings

Analytical conditions: 40° C. (2 min)+2 degrees per minute up to 300° C. (5 minutes).

Apparatus: PerkinElmer Clarus 500. Column BPX5 (25 m, diameter 250 μm). Carrier gas helium 15 ml/min. FID and mass detectors. Injector temperature: 250° C. FID temperature: 260° C. Mass detector temperature: 200° C. Retention time: 8.3 minutes.

### Heck C—C Coupling Reaction

1 ml of system (10⁻² mol/l of Pd) formed of preformed palladium nanoparticles in glycerol is placed in the Schlenk tube under argon. 1.5 mmol of styrene, 2.5 mmol of Na₂CO₃ or t-BuOK and 1 mmol of iodo derivative are successively introduced. The reaction is carried out at 100° C. for 12 h. The solution is then cooled to ambient temperature and the products are extracted with dichloromethane (5×3 ml). The organic phase is filtered through celite, the solvent is evaporated under reduced pressure and the residue is analyzed by GC-MS and ¹H NMR. The products are obtained with yields of 92% and 96%.

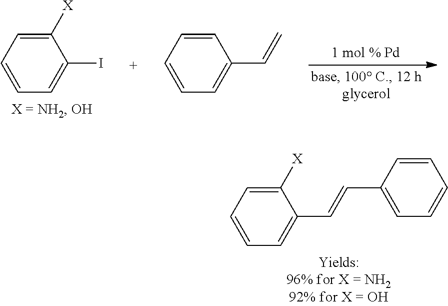

### Sonogashira C—C Coupling Reaction

According to the general protocol, 1 ml of catalytic system (10⁻² mol/l of Pd) formed of preformed palladium nanoparticles in glycerol is placed in a Schlenk tube under argon. 1.5 mmol of alkyne derivative, 2.5 mmol of Na₂CO₃ or t-BuOK and 1 mmol of substrate are successively introduced therein. The reaction is carried out at 80-100° C. for 6 h-24 h. The solution is cooled to ambient temperature and the products are extracted with dichloromethane (5×3 ml). The organic phase is filtered through celite, the solvent is evaporated under reduced pressure and the corresponding residue is analyzed by GC-MS and ¹H NMR.

GC/MS Analyses

Analytical conditions: 40° C. (2 min)+2 degrees per minute up to 300° C. (5 minutes).

Apparatus: PerkinElmer Clarus 500. Column BPX5 (25 m, diameter 250 μm). Carrier gas helium 15 ml/min. FID and mass detectors. Injector temperature: 250° C. FID temperature: 260° C. Mass detector temperature: 200° C. Retention time: 12.1 minutes.

### EXAMPLE 6

### Multistage Reactions Catalyzed by PdNP in Glycerol

The results obtained for the reactions presented in example 5 led to the use of the catalytic system for cascade reactions which make possible the formation of several new C—C bonds in a single reactor (one-pot synthesis), without the need to isolate or purify the intermediate products formed, with the consequent decrease in the cost of the process. The preformed PdNP in glycerol made possible the formation of heterocycles, such as furans, indoles and phthalimides, with high yields.

Three reaction schemes for multistage reactions, two cascade processes (a, b) and a sequential process (c), are presented below by way of example, in which catalysis is carried out by PdNP in glycerol medium:

(a) a Sonogashira coupling, followed by cyclization,

(b) carbonylative couplings, and

(c) a Heck coupling, followed by hydrogenation.

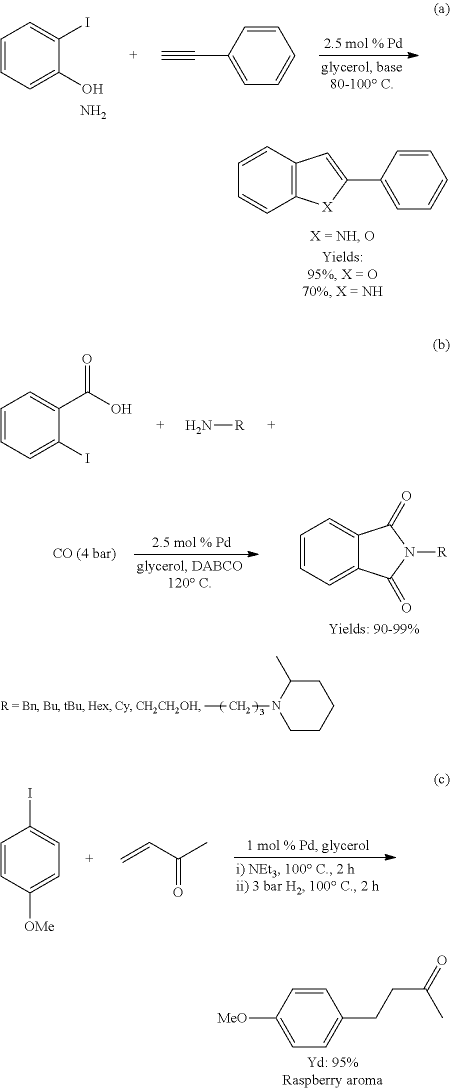

### Experimental Protocol

a) Sonogashira coupling, followed by cyclization: the coupling of phenylacetylene with 2-iodophenol was carried out with 65.8 μl of phenylacetylene (0.6 mmol), 1.0 mmol of t-BuOK (112 mg) and 0.4 mmol of 2-iodophenol (88 mg), using 1 ml of catalytic system (10⁻² mol/l of Pd). The reaction is carried out at 80° C. for 24 h. The solution is cooled to ambient temperature and the products are extracted with dichloromethane (5×3 ml). The organic phase is evaporated under reduced pressure and the residue is purified by flash chromatography with a CH₂Cl₂/hexane=90/10 eluent mixture. The product is analyzed by GC-MS and ¹H NMR. 75 mg of final product are recovered (yield 95%).

b) Carbonylative Coupling

According to the general protocol, 1 ml of catalytic system (10⁻² mol/l of Pd) formed of preformed palladium nanoparticles in glycerol is placed in a Fischer-Porter bottle under argon in the presence of 0.4 mmol of substrate, 0.6 mmol of amine derivative and 1 mmol of DABCO. The reaction is carried out at 120° C. for 30 minutes under 0.5 bar of carbon monoxide. The solution is cooled to ambient temperature and the products are extracted with dichloromethane (5×3 ml). The organic phase is filtered through celite, the solvent is evaporated under reduced pressure and the corresponding residue is analyzed by GC-MS and ¹H NMR. The reaction yield is of the order of 90 to 99%, depending on the amine used. For example, 1 ml of catalytic system (10⁻² mol/l of Pd) formed of preformed palladium nanoparticles in glycerol is placed in a Fischer-Porter bottle under argon in the presence of 0.4 mmol of 2-iodobenzoic acid (99.2 mg), 0.4 mmol of benzylamine (43.7 μl) and 1.2 mmol of DABCO (112 mg). The reaction is carried out at 120° C. under 0.5 bar of carbon monoxide. The solution is cooled to ambient temperature and the products are extracted with dichloromethane (5×3 ml). The organic phase is filtered through celite, the solvent is evaporated under reduced pressure and the residue is analyzed by GC-MS and ¹H NMR. 92 mg of product are recovered (yield 96%).

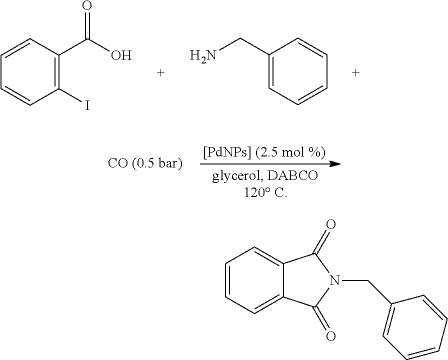 [SMILES: O=C(O)c1ccccc1I ; NCc1ccccc1 ; O=C1c2ccccc2C(=O)N1Cc1ccccc1]

Weights recovered during different recyclings:
first handling 92 mg (97%)
recycling 1: 92 mg (97%) recycling 2: 90 mg (94%)
recycling 3: 93 mg (98%) recycling 4: 92 mg (97%)
recycling 5: 91 mg (95%) recycling 6: 90 mg (94%)
recycling 7: 88 mg (93%) recycling 8: 89 mg (93%)
recycling 9: 90 mg (94%) recycling 10: 91 mg (95%)

### EXAMPLE 7

### Formation of Triazole Compounds Catalyzed by Cu₂ONP/Glycerol

### Synthesis of Compounds Comprising a Heterocycle

Triazoles, in particular the derivatives of 1,2,3-triazoles, are known for their activity against the HIV-1 virus, orthopoxviruses and the SARS (severe acute respiratory syndrome) virus. These compounds are, for example, as follows:

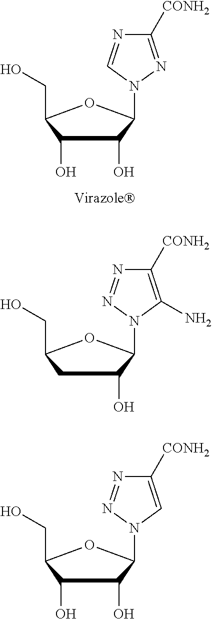

One of the stages in their synthesis is the formation of the triazole ring. The catalytic system Cu₂ONP in glycerol (see example 2) makes it possible to prepare triazoles with high yields. More than twenty compounds have been prepared with different R₁ and R₂ substituents carried by the heterocycle according to the scheme below:

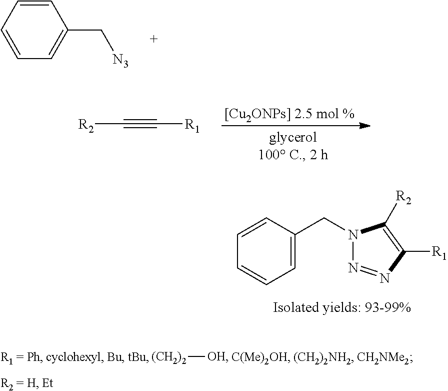

The yields obtained range from 93% to 99% as the case may be. The catalytic phase can be recycled more than ten times without loss of the catalytic properties.

### Synthesis of Compounds Comprising Two or Three Heterocycles

Compounds comprising two or three triazole rings have been obtained, with yields greater than 94%, for example the compounds below:

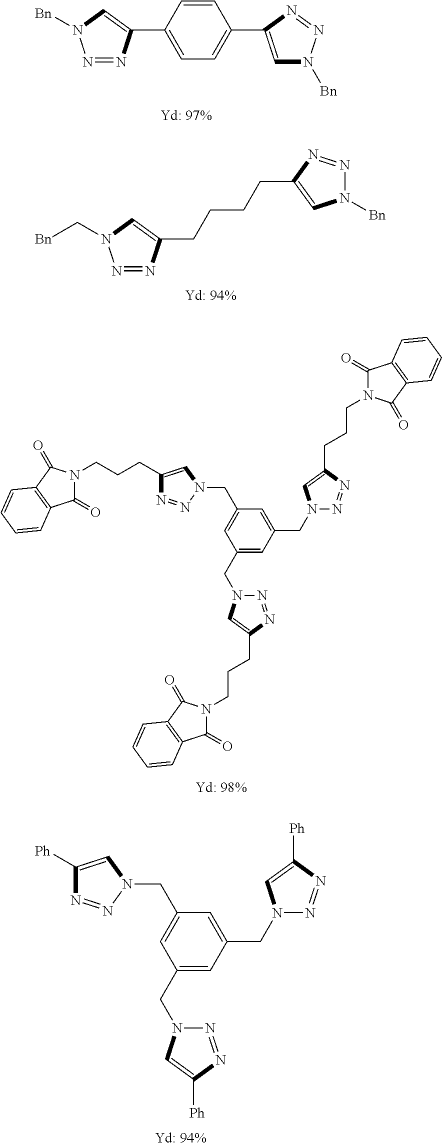

### Cascade Reactions

As the system Cu₂ONP in glycerol makes possible the formation of C—N and C—S bonds, cascade reactions have been carried out. They have made it possible to synthesize the expected products, with yields of more than 90%. This strategy has made it possible to obtain polyfunctional molecules in a one-pot process, without isolating the intermediate products, thus saving in their purification.

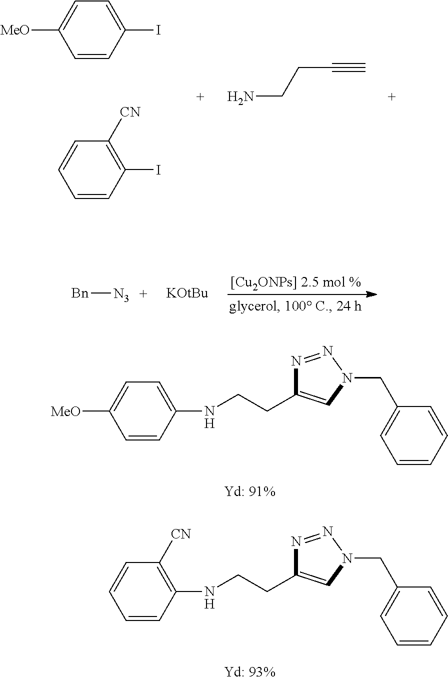

Whatever the operating conditions, the glycerol remained stable and showed no sign of decomposition.

### EXAMPLE 8

### Formation of Heterocycles Catalyzed by PdNP/Glycerol

Benzofurans, isobenzofurans, isoindolinones or phthalimides are heterocycles having pharmacological properties which are often found in natural products. Mention may be made, among these, for example, of the following compounds:

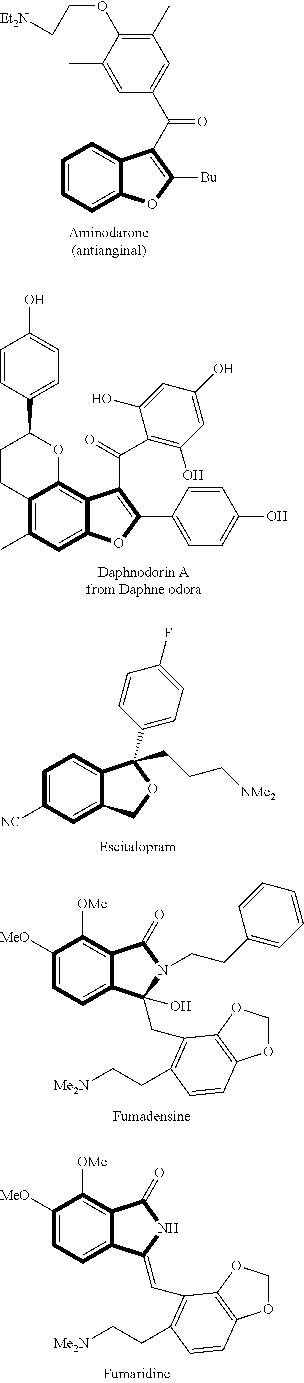

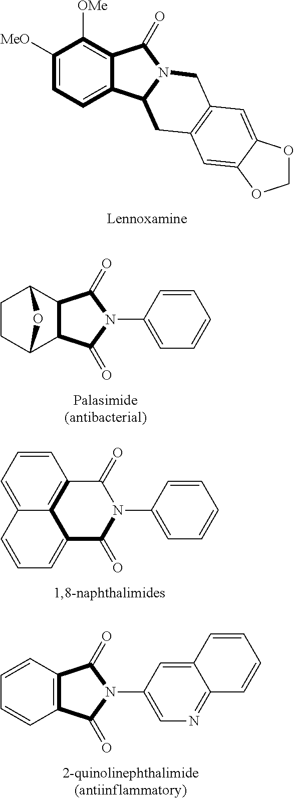

Different types of heterocycles were synthesized by cascade reactions, always in a one-pot process. The yields are high in all the scenarios, a few examples of which are given below:

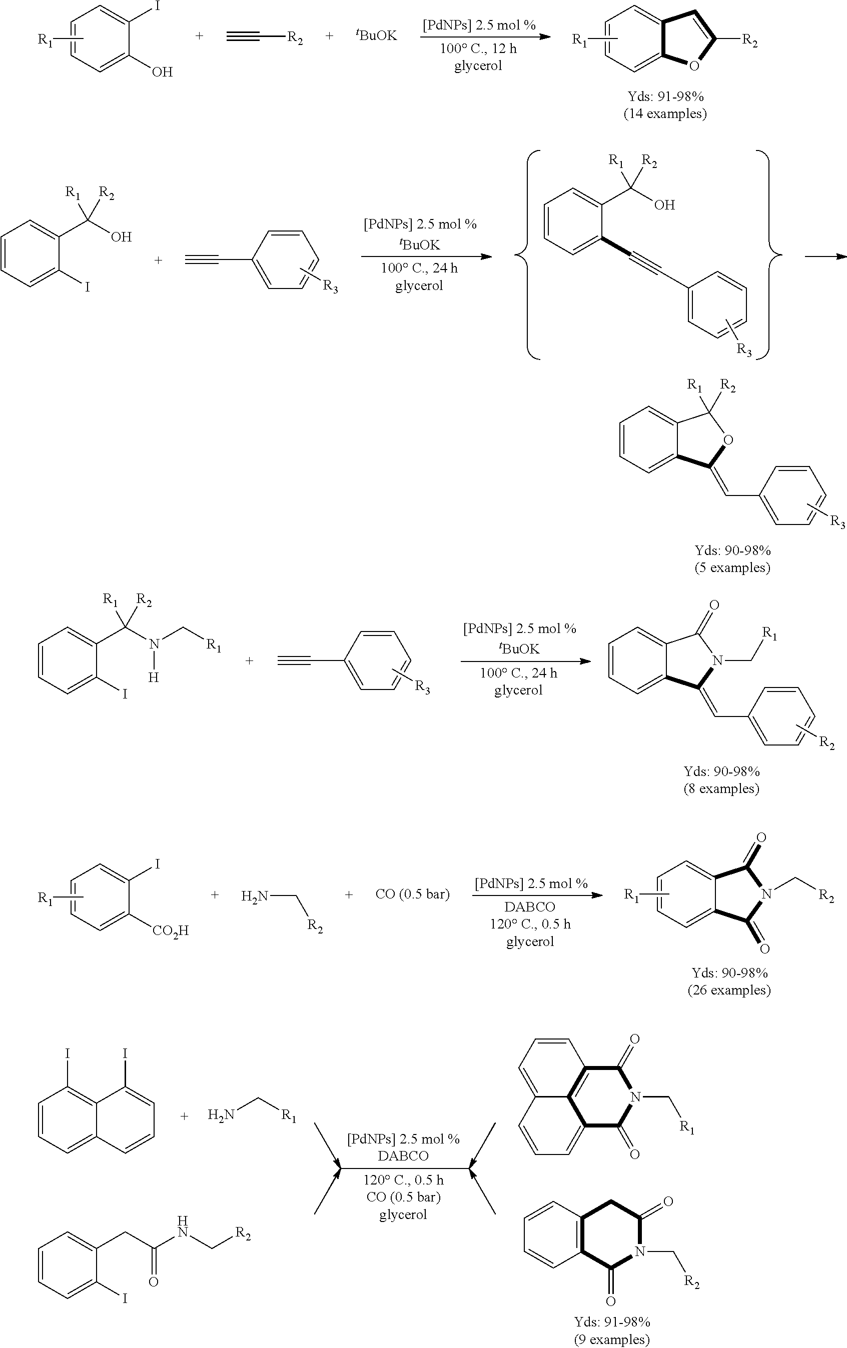

More complex molecules comprising different types of heterocycles could be obtained by a multistage synthesis composed of two consecutive tandem processes, both catalyzed by the palladium/glycerol system:

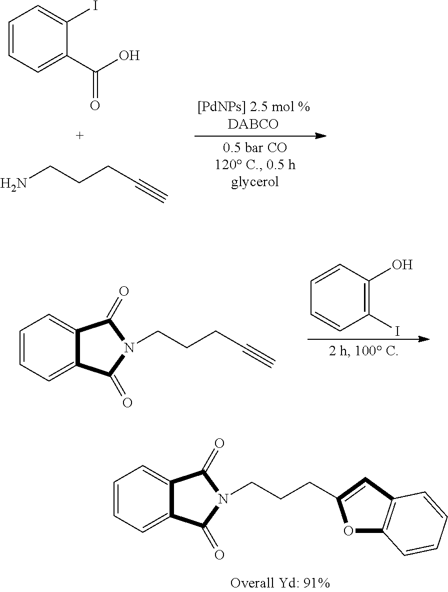

1-19. (canceled)

20. A catalytic system, consisting of a suspension in glycerol of metal nanoparticles comprising at least one transition metal, said suspension also comprising at least one glycerol-soluble stabilizing compound which stabilizes said metal nanoparticles.

21. The system as claimed in claim 20, wherein said nanoparticles comprise a metal having a zero oxidation state chosen from the transition metals from Groups VI to XI.

22. The system as claimed in claim 20, wherein said nanoparticles comprise an oxide of a transition metal having a given oxidation state, said metal being chosen from the metals of the first transition series.

23. The system as claimed in claim 20, wherein said nanoparticles comprise a metal chosen from copper, palladium, rhodium and ruthenium.

24. The system as claimed in claim 20, wherein said stabilizing compound is a ligand of said transition metal chosen from glycerol-soluble phosphines.

25. The system as claimed in claim 24, wherein said stabilizing compound is the sodium salt of tris(3-sulfophenyl)phosphine, with a molar ratio of said ligand to said metal being of between 0.1 and 2.0.

26. The system as claimed claim 20, wherein said transition metal is at a concentration in the glycerol of between 10⁻¹ mol/l and 10⁻⁴ mol/l.

27. A process for obtaining a catalytic system consisting of a suspension in glycerol of metal nanoparticles as claimed in claim 20, comprising the stages consisting essentially in:
a) introducing, into a reactor, i) an amount of glycerol, ii) at least one precursor compound of a transition metal, and iii) at least one glycerol-soluble stabilizing compound which stabilizes said metal nanoparticles;
b) placing this reaction mixture under a pressure of a reducing gas of between 10⁵ Pa and 5×10⁵ Pa, at a temperature of between 30° C. and 100° C., and allowing reaction to take place until a suspension of nanoparticles of said metal compound has formed.

28. The process for obtaining a catalytic system as claimed in claim 27, wherein said precursor is a salt or an organometallic complex of a transition metal belonging to one of Groups VI to XI.

29. The process for obtaining a catalytic system as claimed in claim 27, wherein said transition metal is chosen from copper, palladium, rhodium or ruthenium.

30. The process for obtaining a catalytic system as claimed in claim 27, wherein said stabilizing compound is a ligand of said transition metal chosen from glycerol-soluble phosphines.

31. The process for obtaining a catalytic system as claimed claim 30, wherein said stabilizing compound is the sodium salt of tris(3-sulfophenyl)phosphine, with a molar ratio of said ligand to said metal precursor is of between 0.1 and 2.0.

32. The process for obtaining a catalytic system as claimed claim 27, wherein said metal precursor is introduced into the reactor at a concentration between 10⁻¹ mol/l and 10⁻⁴ mol/l.

33. The process for obtaining a catalytic system as claimed in claim 27, wherein the pressure of reducing gas is produced by molecular hydrogen at 3×10⁵ Pa.

34. The process for obtaining a catalytic system as claimed in claim 27, wherein the reaction temperature in stage b) is of the order of 30° C. to 60° C.

35. A method for catalyzing an organic synthesis reaction starting from a substrate, comprising the steps of:
i) bringing said substrate into contact with a catalytic system as claimed in claim 20 comprising at least one metal capable of catalyzing said reaction, at a temperature of between 30° C. and 100° C.; and
ii) at the end of the reaction, separating the products and the catalytic system.

36. The method as claimed in claim 35, wherein, once the products have been separated, said catalytic system is recycled by subjecting it to a reduced pressure of the order of 10³ Pa and steps i) and ii) are repeated at least once, with identical or different substrates and reactants.

37. The method according to claim 35, wherein said reaction is selected from the group consisting of:
hydrogenation catalyzed by a catalytic system comprising rhodium, palladium or ruthenium nanoparticles, in suspension in glycerol;
a reaction in which the formation of a C—N or C—S bond is catalyzed by a catalytic system comprising copper(I) oxide nanoparticles in suspension in glycerol;
a reaction in which the formation of a C—C bond is catalyzed by a catalytic system comprising palladium nanoparticles in suspension in glycerol; and
a Suzuki C—C cross-coupling reaction, Heck C—C cross-coupling reaction or Sonogashira C—C cross-coupling reaction.

38. The method according to claim 35, wherein several reactions are carried out in a single reactor, in cascade or sequentially, without isolating or purifying the intermediate products.

39. The system as claimed in claim 20, wherein said nanoparticles comprise a mixture of oxides of a transition metal having different oxidation states, said metal being chosen from the metals of the first transition series.
